(19) United States
(12) Patent Application Publication
(10) Pub. No.: US 2010/0249248 A1
(43) Pub. Date: Sep. 30, 2010

(54) LIGAND AGENTS FOR PEROXISOME PROLIFERATOR-ACTIVATED RECEPTORS (PPARs)

(75) Inventors: Kyoichi Ogura, Osaka (JP); Mitsuru Maeda, Osaka (JP); Tomohiro Rogi, Osaka (JP); Yoshinori Kitagawa, Osaka (JP); Kahori Shimada, Osaka (JP)

Correspondence Address: DRINKER BIDDLE & REATH (DC), 1500 K STREET, N.W., SUITE 1100, WASHINGTON, DC, 20005-1209, US

(73) Assignee: Suntory Holdings Limited, Osaka-shi (JP)

(21) Appl. No.: 12/739,568

(22) PCT Filed: Oct. 24, 2008
(86) PCT No.: PCT/JP2008/069363

§ 371 Date: Apr. 23, 2010

(30) Foreign Application Priority Data
Oct. 24, 2007 (JP) 2007-277021

(51) Int. Cl. A61K 31/045; C07C 31/02; A61P 3/10; A61P 3/06; A61P 9/12

(52) U.S. Cl. 514/724; 568/840

(57) ABSTRACT

The present invention aims to provide ligand agents for peroxisome proliferator-activated receptors (PPARs), etc.

The ligand agents of the present invention are characterized by comprising, as an active ingredient, an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

### TECHNICAL FIELD

The present invention relates to ligand agents for peroxisome proliferator-activated receptors (PPARs), which are effective for prevention and/or treatment of PPAR-related diseases. The present invention further relates to pharmaceutical compositions and/or food or beverage products for prevention and/or treatment of the above diseases.

### BACKGROUND ART

Peroxisome proliferator-activated receptors (PPARs) belong to the nuclear receptor family that controls the expression of genes responsible for maintaining lipid and carbohydrate metabolism, and they act as factors which regulate transcription in a ligand-dependent manner. They are known to have three subtypes: PPARα, PPARγ and PPARδ (PPARβ).

PPARα is expressed primarily in the liver, cardiac muscle or the like and regulates lipid metabolism. In particular, PPARα is found to be highly expressed in the liver. Ligands known for PPARα include synthetic compounds such as fatty acids (e.g., palmitic acid, oleic acid, linolic acid, arachidonic acid) and fibrate-type hypolipidemic drugs (e.g., bezafibrate, clofibrate). These ligands are known to activate liver PPARα to stimulate lipid metabolism, thereby producing a hypolipidemic effect in blood (Non-patent Documents 1 and 2).

PPARγ is expressed primarily in fat tissue and is involved in differentiation of small adipocytes. Moreover, PPARγ also induces apoptosis of large adipocytes showing enhanced production and secretion of TNFα or free fatty acid responsible for insulin resistance, so that PPARγ improves insulin resistance and thereby has a hypoglycemic effect or the like. Ligands known for PPARγ include synthetic compounds such as unsaturated fatty acids (e.g., α-linolenic acid, eicosapentaenoic acid, docosahexaenoic acid) and thiazolidine-type antidiabetic drugs (e.g., troglitazone, pioglitazone, rosiglitazone). These ligands are known to suppress hyperplasia of large adipocytes and to increase the number of insulin-sensitive small adipocytes, so that they improve insulin resistance and thereby reduce blood glucose levels (Non-patent Documents 1 and 2).

Since PPARδ is expressed in a non-tissue-specific manner, its functions are difficult to predict and hence its physiological actions have remained unknown. However, recent studies have indicated that PPARδ is highly expressed in skeletal muscle cells, and further that PPARδ is involved in the expression of genes associated with fatty acid metabolism and has the function of stimulating fatty acid metabolism in skeletal muscle cells or fat tissue. Moreover, PPARγ ligands have been found to not only suppress high-fat diet-induced body weight gain in obesity mouse models, but also to have an improving effect on insulin resistance. Furthermore, in transgenic mice designed to overexpress PPARδ in their skeletal muscle, it has been indicated that these mice are less likely to develop high-fat diet-induced obesity or insulin resistance, and their adipocytes become smaller in size (Non-patent Document 1).

Ligands reported for PPARδ include polyunsaturated fatty acids (e.g., dihomo-γ-linolenic acid, arachidonic acid, eicosapentaenoic acid), eicosanoids (e.g., prostaglandin A1, prostaglandin D2), as well as a semi-synthetic prostaglandin, carbacyclin (Non-patent Document 2).

Other PPAR ligands reported are polyphenols such as gallic acid esters, galloyltannins, quercetins, flavone, isoflavone, catechin and epicatechin (Patent Document 1).

Patent Document 1: Japanese Patent Laid-Open Publication 2002-80362 A

Patent Document 2: Japanese Patent Laid-Open Publication 2003-95968 A

Patent Document 3: Japanese Patent Laid-Open Publication 2006-42816 A

Patent Document 4: Japanese Patent Laid-Open Publication 2003-26694 A

Patent Document 5: Japanese Patent Laid-Open Publication 2005-8572 A

Patent Document 6: Japanese Patent Laid-Open Publication 2006-16330 A

Patent Document 7: Japanese Patent Laid-Open Publication 2005-126405 A

Patent Document 8: Japanese Patent Laid-Open Publication 2006-20606 A

Patent Document 9: Japanese Patent Laid-Open Publication 2006-230225 A

Patent Document 10: Japanese Patent Laid-Open Publication 2006-22095 A

Patent Document 11: Japanese Patent Laid-Open Publication 2006-520804 A

Patent Document 12: WO 97/28149

Non-patent Document 1: Japanese Journal of Clinical Medicine, 2005, vol. 63, pp. 557-583

Non-patent Document 2: Journal of Medicinal Chemistry, 2000, vol. 43, pp. 527-550

Non-patent Document 3: Journal of Periodontology, 2006, vol. 77, pp. 271-279

Non-patent Document 4: Phytother. Res., 2007, vol. 21, pp. 391-394

Non-patent Document 5: Journal of Agricultural and Food Chemistry, 54, 335-341 (2006)

Non-patent Document 6: Annals of Medicine, 2005, vol. 37, pp. 270-275

Non-patent Document 7: Exp Biol Med, 2005, vol. 230, pp. 225-234

### DISCLOSURE OF THE INVENTION

### Problems to be Solved by the Invention

As described above, PPAR ligands (herein also referred to as “PPAR ligand agents”) are expected to prevent and/or ameliorate obesity and obesity-associated insulin resistance, as well as hyperlipidemia, hypertension, diabetes and so on because of their ability to enhance lipid and/or carbohydrate metabolism. However, existing PPAR ligands are feared to cause side effects when administered long-term as pharmaceutical preparations, while food-derived polyphenols or the like do not have sufficient PPAR ligand action (herein also referred to as “ligand activity”). Thus, their potency has not always been satisfactory as PPAR ligand agents.

The object of the present invention is to provide safe PPAR ligand agents having excellent PPAR ligand action.

### Means for Solving the Problems

To obtain PPAR ligands that are safe for long-term intake and have fewer side effects, the inventors of the present invention have made extensive and intensive efforts to search for PPAR ligands among various plant extracts. As a result, the inventors have succeeded in obtaining specific edible plant extracts having excellent PPAR ligand action. Moreover, the inventors have found that properties of their ligand action would vary depending on the type of solvent, etc. This finding led to the completion of the present invention.

The present invention encompasses the following as preferred embodiments.

### Embodiment 1

A ligand agent for a peroxisome proliferator-activated receptor (PPAR), which comprises, as an active ingredient, an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

### Embodiment 2

The ligand agent according to Embodiment 1, wherein the PPAR is PPARδ.

### Embodiment 3

The ligand agent according to Embodiment 1 or 2, wherein the plant is one or more selected from Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

### Embodiment 4

The ligand agent according to any one of Embodiments 1 to 3, wherein the extract is an alcohol extract or an aqueous alcohol extract.

### Embodiment 5

The ligand agent according to Embodiment 4, wherein the alcohol is ethanol.

### Embodiment 6

The ligand agent according to Embodiment 1 or 2, wherein the plant is one or more selected from Linum usitatissimum, Pleurotus ferulae, Acanthopanax sessiliflorus, and Cannabis sativa.

### Embodiment 7

The ligand agent according to Embodiment 1, 2 or 6, wherein the extract is a hot water extract.

### Embodiment 8

A pharmaceutical composition used for prevention and/or treatment of a peroxisome proliferator-activated receptor-related disease, which comprises an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus and a pharmaceutically acceptable additive.

### Embodiment 9

The pharmaceutical composition according to Embodiment 8, wherein the peroxisome proliferator-activated receptor-related disease is one or more diseases selected from obesity, hyperlipidemia, hypertension and diabetes.

### Embodiment 10

A food or beverage product, which comprises an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus and an additive acceptable for food or beverage purposes.

### Embodiment 11

The food or beverage product according to Embodiment 10, which is used for prevention and/or amelioration of a peroxisome proliferator-activated receptor-related disease.

### Embodiment 12

An anti-obesity agent, which comprises an extract of one or more plants selected from the group consisting of Sambucus nigra, Pimpinella anisum, Crataegus monogyna and Adinandra nitida as an active ingredient.

### Embodiment 13

A method for prevention and/or treatment of a peroxisome proliferator-activated receptor-related disease, which comprises administering to a subject in need of such prevention and/or treatment, a pharmaceutical composition comprising an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

### Embodiment 14

Use for the manufacture of a pharmaceutical composition used for prevention and/or treatment of a peroxisome proliferator-activated receptor-related disease, wherein the pharmaceutical composition comprises an extract of one or more plants selected from the following:

Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

### Embodiment 15

A method for prevention and/or treatment of obesity, which comprises administering to a subject in need of such prevention and/or treatment, an anti-obesity agent comprising an extract of one or more plants selected from the group consisting of Sambucus nigra, Pimpinella anisum, Crataegus monogyna and Adinandra nitida.

### Embodiment 16

Use for the manufacture of an anti-obesity agent used for prevention and/or treatment of obesity, wherein the anti-obesity agent comprises an extract of one or more plants selected from the group consisting of Sambucus nigra, Pimpinella anisum, Crataegus monogyna and Adinandra nitida.

### ADVANTAGES OF THE INVENTION

According to the present invention, there are provided excellent PPAR ligand agents and compositions (pharmaceutical compositions, food or beverage products) comprising the same as an active ingredient.

Plant extracts serving as active ingredients in the ligand agents of the present invention are acceptable for long-term intake because they have been taken as naturally-occurring foods and are highly safe. Thus, when taken daily in the form of oral compositions, these extracts can be used as prophylactic compositions for peroxisome proliferator-activated receptor-related diseases including obesity, hyperlipidemia, hypertension, diabetes, etc. Moreover, these extracts can also be used as therapeutic and ameliorative compositions for the above diseases in need of long-term treatment and amelioration.

### BEST MODE FOR CARRYING OUT THE INVENTION

### I. PPAR ligand agents

The present invention relates to ligand agents for peroxisome proliferator-activated receptors (PPARs) and/or anti-obesity agents, each of which comprises a specific plant extract(s) as an active ingredient.

(Plant Extracts)

Plants used as source materials for the ligand agents of the present invention are one or more edible plants selected from the group consisting of the 23 plants listed below:

(1) Sambucus nigra;

(2) Adinandra nitida;

(3) Passiflora incarnata;

(4) Peucedanum japonicum;

(5) Pimpinella anisum;

(6) Vitex agnus-castus ;

(7) Withania somnifera;

(8) Calluna vulgaris;

(9) Crataegus monogyna;

(10) Linum usitatissimum;

(11) Pleurotus ferulae;

(12) Robinia pseudoacacia;

(13) Acanthopanax sessiliflorus;

(14) Cannabis sativa;

(15) Elettaria cardamomum;

(16) Angelica archangelica;

(17) Psidium guajava;

(18) Prumus spinosa;

(19) Serenoa repens;

(20) Eleagnus multiflora;

(21) Ligusticum chuaxiong;

(22) Acanthopanax senticosus; and

(23) Euterpe oleracea (or E. edulis).

Explanation will be given below for each plant.

(1) Sambucus nigra: This is a plant of the family Caprifoliaceae, and its flowers are preferred for use in the present invention. Flowers of Sambucus nigra, which are also called elderflowers, are a herb that has been used in Europe since a long time, and are known to be effective for amelioration of common cold, sore throat, arthritis, etc. Moreover, there is a report of their suppressive effect on early inflammation in periodontal disease (Non-patent Document 3). However, it is completely unknown that a solvent extract of Sambucus nigra serves as a PPAR ligand agent.

(2) Adinandra nitida: This is a plant of the family Theaceae, and its leaves are preferred for use in the present invention. Adinandra nitida, which is a special tea product of the Guangxi Zhuang autonomous region of southern China, is believed to have anti-inflammatory and antidotal action, to be good for sore throat and inflammation of mouth, and also to have a hypotensive effect and a prophylactic effect on cancer. However, it is completely unknown that a solvent extract of Adinandra nitida serves as a PPAR ligand agent.

(3) Passiflora incarnata: This is a plant of the family Passifloraceae, and its terrestrial part is preferred for use in the present invention. The terrestrial part of Passiflora incarnata is also called passionflower and is a herb used for neurotic anxiety and insomnia in Europe. A prophylactic composition for cardiovascular disease is known, which comprises one or more of several polyphenol materials including Passiflora incarnata (passionflower) together with a garlic material (Patent Document 2). Moreover, there are disclosed dietary fiber-containing diet foods designed with the aim of reducing mental stress during the diet period, among which is a diet food comprising passionflower (Patent Document 3). However, none of these documents mentions the physiological action of Passiflora incarnata alone, nor do they suggest or disclose that a solvent extract of Passiflora incarnata serves as a PPAR ligand agent.

(4) Peucedanum japonicum: This is also called “botanboufuu” in Japan and is a plant of the family Umbelliferae. Its terrestrial part is preferred for use in the present invention. Patent Document 4 shows that a dry powder or extract of Peucedanum japonicum has an inhibitory activity on disaccharide-degrading enzymes and is effective for anti-diabetes and anti-obesity purposes. However, it is completely unknown that a solvent extract of Peucedanum japonicum serves as a PPAR ligand agent.

(5) Pimpinella anisum: This is a plant of the family Umbelliferae, and its seeds are preferred for use in the present invention. Seeds of Pimpinella anisum are used as an aromatic herb in beverages, baked sweets, herbal teas, etc. They are also used as a folk medicine for diuresis, dyspepsia, bronchitis, etc. However, it is completely unknown that a solvent extract of Pimpinella anisum serves as a PPAR ligand agent.

(6) Vitex agnus-castus: This is a plant of the family Verbenaceae, and its fruits are preferred for use in the present invention. In Europe, fruits of Vitex agnus-castus have been used as a spice and also as a folk medicine for menoxenia and premenstrual tension. Moreover, there is a report on the antioxidative activity of flavonoids contained in an ethanol extract of this plant (Non-patent Document 4). However, it is completely unknown that a solvent extract of Vitex agnus-castus serves as a PPAR ligand agent.

(7) Withania somnifera: This is a plant of the family Solanaceae, and its roots are preferred for use in the present invention. Leaves or roots of Withania somnifera have been used as a longevity drug “Sarayan” in Ayurveda (Indian traditional medication) since a long time. This plant is known to be efficacious for nutrition and tonic purposes, as well as aphrodisiac, anti-anxiety and anti-depression purposes, and also efficacious for alleviating arthritis such as rheumatism. However, it is completely unknown that a solvent extract of Withania somnifera serves as a PPAR ligand agent.

(8) Calluna vulgaris: This is a plant of the family Ericaceae, and its flowers are preferred for use in the present invention. In Europe, flowers of Calluna vulgaris are used as a herb effective for prevention of urinary tract infection and effective against diuresis, arthritis, insomnia, respiratory diseases, etc. Their leaves and stems are also used as health tea ingredients having the same effects as above. Patent Document 5 discloses the anti-obesity effect and acne-ameliorating effect of this plant when used as a fat absorption inhibitor based on lipase inhibition. However, these effects are distinct from the intended lipid metabolism-improving effect as a PPAR ligand agent. Thus, it is completely unknown that a solvent extract of Calluna vulgaris serves as a PPAR ligand agent.

(9) Crataegus monogyna: This is a plant of the family Rosaceae, and its fruits are preferred for use in the present invention. In Europe, flowers, leaves and fruits of Crataegus monogyna are used as herbs effective against heart disease, and particularly the fruits are used as a herb effective against arteriosclerosis and kidney disease. However, it is completely unknown that a solvent extract of Crataegus monogyna serves as a PPAR ligand agent.

(10) Linum usitatissimum: This is a plant of the family Linaceae, and its seeds are preferred for use. Seeds of Linum usitatissimum are rich in ω3 fatty acids and are known to have a hypocholesterolemic effect in blood. Moreover, in Finland where people take Linum usitatissimum seeds, it is epidemiologically known that hormone-sensitive cancers (e.g., prostate cancer, breast cancer) occur at low incidence rates. As described above, it is known that fatty acids serve as PPAR ligand agents, but there is no actual disclosure or suggestion that a solvent extract of Linum usitatissimum serves as a PPAR ligand agent.

(11) Pleurotus ferulae: This is a plant of the family Tricholomataceae, and its fruit body is preferred for use in the present invention. This plant is known to have an antitumor effect which enhances immunity, and is also known to be effective against diabetes by enhancing pancreatic functions. However, it is completely unknown that a solvent extract of Pleurotus ferulae serves as a PPAR ligand agent.

(12) Robinia pseudoacacia: This is a plant of the family Leguminosae, which is also called false acacia, and its flowers are preferred for use in the present invention. In traditional Chinese medicines, flowers of this plant are used for melena and hemoptysis. Patent Document 6 discloses fruits of false acacia as a material related to lipid burning stimulators comprising proanthocyanidin as an active ingredient. However, parts preferred for use in this case differ from those of the present invention, and there is no suggestion or disclosure about use as a PPAR ligand agent.

(13) Acanthopanax sessiliflorus: This is a plant of the family Araliaceae, and its leaves are preferred for use in the present invention. The root bark of Acanthopanax sessiliflorus is used as a traditional Chinese medicine “Wujiapi” for anti-inflammatory, analgesic and tonic purposes, while leaves of this plant are used for edible purposes or as a health tea ingredient. Non-patent Document 5 reports that saponin in leaves of Acanthopanax sessiliflorus inhibits pancreatic lipase to thereby produce an anti-obesity effect. However, this effect is distinct from the intended lipid metabolism-improving effect as a PPAR ligand agent. Thus, it is completely unknown that a solvent extract of Acanthopanax sessiliflorus serves as a PPAR ligand agent.

(14) Cannabis sativa: This is a plant of the family Cannabaceae, and its seeds are preferred for use. There are reports showing that a neurotropic substance, cannabinoid, which is contained in leaves and/or corollas of Cannabis sativa acts on the nervous system and thereby participates in appetite and energy metabolism (Non-patent Documents 6 and 7). However, it is not known that fruits (seeds) of Cannabis sativa stimulate lipid metabolism. Moreover, it is completely unknown that a solvent extract of Cannabis sativa serves as a PPAR ligand agent.

(15) Elettaria cardamomum: This is a plant of the family Zingiberaceae, and its seeds are preferred for use in the present invention. Seeds of Elettaria cardamomum are used as a spice. Patent Document 7 discloses an anti-obesity agent characterized by comprising water-soluble galactomannan, a food emulsifier, yeast powder and vitamins B, and it also discloses that an essential oil of Elettaria cardamomum is incorporated into this anti-obesity agent. However, the major effect of this patent is to prevent obesity by the actions of water-soluble galactomannan to suppress appetite through stomach distension and to reduce or delay glucose and fat absorption. This document shows, but does not prove, that the essential oil stimulates body metabolisms when incorporated into the agent. Also, there is no suggestion or disclosure that a solvent extract of Elettaria cardamomum serves as a PPAR ligand agent.

(16) Angelica archangelica: This is a plant of the family Umbelliferae, and its roots are preferred for use in the present invention. In Europe, roots of Angelica archangelica are a herb used for symptoms such as loss of appetite, dyspepsia, and distension. Angelica archangelica is known to be efficacious for hypertension, anaemia, diabetes, neuralgia, diuresis, fatigue recovery, thrombosis prevention, etc. However, it is completely unknown that this plant serves as a PPAR ligand agent.

(17) Psidium guajava: This is a plant of the family Myrtaceae, and its immature fruits are preferred for use. Patent Documents 8 and 9 disclose the prophylactic and ameliorative effects of Psidium guajava tea on obesity. However, these effects lie in controlling absorption of fats, starches and sugars through inhibition of digestive enzymes for carbohydrates and fats in Patent Document 8, and rely on suppression of sugar absorption due to inhibitory effects on digestive enzymes for carbohydrates in Patent Document 9. It is not known that immature fruits of Psidium guajava stimulate lipid metabolism. Moreover, it is completely unknown that a solvent extract of Psidium guajava serves as a PPAR ligand agent.

(18) Prumus spinosa: Fruits of this plant are preferred for use in the present invention. In Europe, fruits of Prumus spinosa are used as a herbal tea ingredient and also used as a folk medicine for inflammation in the oral cavity and pharynx mucosa. However, it is completely unknown that a solvent extract of Prumus spinosa serves as a PPAR ligand agent.

(19) Serenoa repens: This is a plant of the family Arecaceae, and its fruits are preferred for use in the present invention. Fruits of Serenoa repens have an anti-androgenic effect and are known to be effective against prostate disorders. However, it is completely unknown that a solvent extract of Serenoa repens serves as a PPAR ligand agent.

(20) Eleagnus multiflora: This is a plant of the family Elaeagnaceae, and its fruits are preferred for use in the present invention. Fruits of Eleagnus multiflora are referred to as “mu ban xia” in traditional Chinese medicine, and are used for treatment of bruises and rheumatic arthralgia. However, it is completely unknown that a solvent extract of Eleagnus multiflora serves as a PPAR ligand agent.

(21) Ligusticum chuaxiong: This is a plant of the family Umbelliferae, and its terrestrial part is preferred for use in the present invention. Rhizomes of this plant are referred to as “chuan xiong” in traditional Chinese medicine and are used as a tonic, sedative or analgesic drug. However, it is completely unknown that a solvent extract of Ligusticum chuaxiong serves as a PPAR ligand agent.

(22) Acanthopanax senticosus: This is a plant of the family Araliaceae, and its root bark is preferred for use in the present invention. The root bark of this plant is used as “Wujiapi” for anti-inflammatory, analgesic and tonic purposes, while its leaves have an anti-stress effect and are used as a health tea ingredient. Patent Document 10 discloses the effects of saponin in leaves of Acanthopanax senticosus (e.g., lipid absorption inhibition, obesity prevention, hyperlipidemia amelioration) based on its inhibitory activity against lipases. However, it is not known that the root bark of Acanthopanax senticosus stimulates lipid metabolism. Moreover, it is completely unknown that a solvent extract of Acanthopanax senticosus serves as a PPAR ligand agent.

(23) Euterpe oleracea: This is also called “wakaba kyabetsu (assai palm)” in Japan and is a plant of the family Arecaceae. Its fruits are preferred for use in the present invention. Fruits of this plant are rich in polyphenols having an antioxidative effect, as well as in amino acids and essential fatty acids. Although there are findings about dietary supplements based on Euterpe edulis (Jucara) and Euterpe oleracea fruits having an antioxidative effect (Patent Document 11), no information can be found on lipid metabolism and prevention or amelioration of obesity. Moreover, it is completely unknown that a solvent extract of Euterpe oleracea serves as a PPAR ligand agent.

Parts of individual plants used for obtaining plant extracts for use in the present invention are preferably those described above, but are not limited thereto. It is also possible to use all parts including buds, flowers, fruits, pericarps, seeds, leaves, branches, stems, bark, roots, root bark, terrestrial parts, and whole plants. In addition to the preferred parts mentioned above, one or more additional parts selected from all of these parts may be used in combination.

Plants for use as source materials may be fresh or dry and, if necessary, may further be processed by grinding, fine cutting, powdering or the like before use. In the case of plants available as crude drugs, such crude drugs may be used.

Plant extracts include those obtained from these various parts by direct extraction with a solvent, as well as those obtained by steam distillation or by carbon dioxide extraction using supercritical extraction technology, those obtained from pressed juices (squeezed juices) and/or their residues by extraction with a solvent, and such pressed juices per se, all of which fall within the definition of a plant extract in the present invention.

The inventors of the present invention have also obtained aqueous alcohol extracts from the above 23 plants by extraction with an aqueous alcohol solution, and have further added ethyl acetate, n-butanol or water to these extracts, followed by liquid-liquid partition to obtain an ethyl acetate, butanol or water fraction for each extract, thereby confirming that one or more of these fractions have excellent PPAR ligand activity. The plant extracts of the present invention also encompass such fractions of solvent extracts.

The plant extracts of the present invention may be subjected to column chromatography or the like to fractionate and purify their active ingredients, if necessary.

(Extraction Procedures)

In the present invention, the above plant extracts or purified products thereof may be used, either alone or in combination.

Extraction solvents used to obtain plant extracts are not limited in any way, and examples include water, lower alcohols containing 1 to 4 carbon atoms (e.g., methanol, ethanol, propanol, butanol), liquid polyhydric alcohols (e.g., 1,3-butylene glycol, propylene glycol, glycerine), ketones (e.g., acetone, methyl ethyl ketone), and esters (e.g., ethyl acetate, butyl acetate). These solvents may be used either alone or in combination.

In a case where the plant used as a source plant is (1) Sambucus nigra (particularly flowers), (2) Adinandra nitida (particularly leaves), (3) Passiflora incarnata (particularly terrestrial part), (4) Peucedanum japonicum (particularly terrestrial part), (5) Pimpinella anisum (particularly seeds), (6) Vitex agnus-castus (particularly fruits), (7) Withania somnifera (particularly roots), (8) Calluna vulgaris (particularly flowers), (9) Crataegus monogyna (particularly fruits), (10) Linum usitatissimum (particularly seeds), (11) Pleurotus ferulae (particularly fruit body), (12) Robinia pseudoacacia (particularly flowers) or (13) Acanthopanax sessiliflorus (particularly leaves), it is preferable to use an alcohol, particularly a lower alcohol or an aqueous solution thereof, as a solvent.

When an alcohol or an aqueous solution thereof is used as a solvent, the alcohol concentration may be selected as appropriate for the desired ligand action. In general, the concentration of an alcohol in an extraction solvent is about 10% to 100% by volume, preferably about 10% to 70% by volume. Since the ligand agents of the present invention are preferred for use as oral compositions such as pharmaceutical compositions or food or beverage products, it is preferable to use ethanol as an alcohol in terms of safety.

Moreover, in a case where the plant used as a source plant is (10) Linum usitatissimum (particularly seeds), (11) Pleurotus ferulae (particularly fruit body) or (13) Acanthopanax sessiliflorus (particularly leaves), it is also preferable to use hot water as a solvent, in addition to the above alcohols or aqueous alcohol solutions. Likewise, in a case where the plant used as a source plant is (14) Cannabis sativa (particularly seeds), it is also preferable to use hot water as a solvent. The term “hot water” as used herein specifically refers to water at 50° C. to 100° C., more preferably water at 50° C. to 85° C.

The above solvents, i.e., hot water, alcohols or aqueous alcohol solutions may further contain any other ingredients, as long as the PPAR ligand action, which is a feature of the present invention, and/or the extraction efficiency is not affected greatly.

Extraction is not limited in any way and may be accomplished by contacting a solvent with such a plant source material as listed above. More specifically, the extraction mode may be determined as appropriate for the desired purpose according to known means, for example, a source material may be immersed and allowed to stand in a solvent or may be stirred or heated under reflux in a solvent. The extraction temperature is not limited in any way and may be determined as appropriate depending on the temperature of a solvent, but it is preferably set below the boiling point of the solvent in terms of handling. If necessary, it is also possible to determine other conditions such as elevated or reduced pressure conditions.

The extraction time may be determined as appropriate depending on the type of plant source material to be used and/or the type and amount of extraction solvent to be used, etc. More specifically, in a case where an alcohol or an aqueous alcohol solution is used as a solvent, it is generally used in an amount of 1- to 1000-fold, preferably 1- to 100-fold, more preferably 1- to 10-fold, relative to 1 part by weight of the source material, while the extraction time is generally about 10 minutes to around 1 month, and preferably 10 minutes to around 7 days. Likewise, in a case where hot water is used as a solvent, it is generally used in an amount of 1- to 1000-fold, preferably 1- to 100-fold, more preferably 1- to 10-fold, relative to 1 part by weight of the source material, while the extraction time is generally about 10 minutes to around 7 days, preferably 10 minutes to around 1 day, and more preferably 10 minutes to around 1 hour.

After the above extraction process, the extraction residue is removed by separation to give the plant extract of the present invention in an extracted solution form. For this separation purpose, any known means may be used, including filtration and centrifugation. In the present invention, the above extracted solution may be used directly or, if necessary, may be used as a concentrated or dried product (concentrated-to-dryness product) of the extracted solution. It is preferably converted into a concentrated or dried product in terms of easy transport, etc. Concentration may be performed under normal pressure or reduced pressure, whereby the volume of a concentrated solution is desirably reduced to about 10% to 50% by volume, preferably about 10% to 30% by volume. To obtain a concentrated-to-dryness product, the solvent may be dried from the extracted solution containing a PPAR ligand active ingredient preferably under reduced pressure.

As described above, the plant extracts of the present invention also include fractions (e.g., ethyl acetate fractions, butanol fractions, water fractions) of extracts obtained by contacting the above plant source materials with a solvent. Such a fraction is generally obtained by liquid-liquid partition from an alcohol or aqueous alcohol extract of a plant source material. Techniques for liquid-liquid partition are not limited in any way, and any known technique may be used for this purpose.

(PPAR Ligand Agents and Anti-Obesity Agents)

As described above, the PPAR ligand agents and anti-obesity agents of the present invention comprise, as an active ingredient, a solvent extract of one or more plants selected from Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

The term “PPAR ligand agent” is used herein as a generic name for agents having the ability to bind to the PPAR ligand-binding region, i.e., having PPAR ligand action. The ligand agents may be either agonists or antagonists, preferably agonists.

The PPAR ligand agents of the present invention can up- or down-regulate the expression of various genes involved in lifestyle-related diseases such as insulin resistance, hyperlipidemia, diabetes, hypertension and obesity. Examples of these various genes include, but are not limited to, those for acyl-CoA oxidase, medium chain acyl-CoA dehydrogenase, carnitine palmitoyltransferase, long chain acyl-CoA synthetase, fatty acid binding proteins, lipoprotein lipase, apolipoproteins and uncoupling proteins.

PPAR ligand activity can be measured, for example, by reporter assay in which binding to a fusion protein between PPAR ligand binding region and GAL4 is represented by luciferase expression (Cell, 1995, vol. 83, pp. 803-812) or by competition binding assay in which a protein containing the PPAR ligand binding region is used (Cell, 1995, vol. 83, pp. 813-819). In these assays, samples are generally compared to a vehicle control, and a sample showing greater activity than the vehicle control is assessed as “having PPAR ligand activity.” In the present invention, an agent showing 1.3-fold or greater activity than a vehicle control is assessed as “having PPAR ligand activity.”

The PPAR ligand agents of the present invention may have ligand action for at least one or more of PPARα, PPARγ and PPARδ. As will be shown in the EXAMPLE section described later, the PPAR ligand agents of the present invention are particularly useful as ligand agents for PPARδ.

Extracts derived from the 23 plants, which can be used in the present invention, each have ligand activity at least for the PPARδ subtype and hence are useful as ligand agents for PPARδ. As described in the BACKGROUND ART section above, physiological actions of PPARδ have remained unknown until recently. With respect to PPARS, agonists capable of activating PPARδ are expected to produce the effect of elevating HDL cholesterol levels to thereby suppress the progression of arteriosclerosis or treat this disease, and are also expected to be used as hypolipidemic or hypoglycemic agents. However, there is no agent that has sufficient activity and is used clinically. Thus, the PPAR ligand agents of the present invention are promising as PPARδ agonists, particularly as hypolipidemic or anti-obesity agents.

Moreover, as demonstrated in the EXAMPLE section described later, the plant extracts have or do not have ligand activity for PPARs of the α- and γ-subtypes, depending on the type of source plant. More specifically, a water fraction of Elettaria cardamomum, as well as ethyl acetate and n-butanol fractions of Linum usitatissimum showed PPARα activity (Example 2). On the other hand, ethyl acetate fractions of Acanthopanax senticosus, Eleagnus multiflora, Cannabis sativa, Sambucus nigra, Adinandra nitida, Pimpinella anisum and Calluna vulgaris, as well as a n-butanol fraction of Adinandra nitida showed PPARγ activity (Example 4). According to the disclosure of the present invention, those skilled in the art would prepare a desired ligand agent when a plant material to be used is selected as needed. Preferably, the PPAR ligand agents of the present invention show ligand activity for both PPARδ and PPARγ.

Moreover, as described later in Example 6, when administered to mice for 16 days, the anti-obesity agents of the present invention were found to have the effects of suppressing body weight gain, suppressing body fat accumulation and lowering blood neutral lipid levels. Thus, the anti-obesity agents of the present invention can also be used as agents for suppressing body weight gain, suppressing body fat accumulation or lowering blood neutral lipid levels, etc. The anti-obesity agents of the present invention preferably comprise an extract of one or more plants selected from the group consisting of Sambucus nigra, Pimpinella anisum, Crataegus monogyna and Adinandra nitida.

More specifically, assuming that the body weight of the untreated control group at the completion of testing was set to 100%, body weight gain was suppressed to about 92% in the group receiving a Sambucus nigra extract (300 mg/kg/day), about 93% in the group receiving a Pimpinella anisum extract (1000 mg/kg/day), about 94% in the group receiving a Crataegus monogyna extract (1000 mg/kg/day) and about 69% in the group receiving an Adinandra nitida extract (100 mg/kg/day). Likewise, assuming that the body fat level of the untreated control group at the completion of testing was set to 100%, body fat accumulation was suppressed to about 81% in the group receiving a Sambucus nigra extract (300 mg/kg/day), about 88% in the group receiving a Pimpinella anisum extract (1000 mg/kg/day), about 80% in the group receiving a Crataegus monogyna extract (1000 mg/kg/day) and about 88% in the group receiving an Adinandra nitida extract (100 mg/kg/day). Moreover, assuming that the blood neutral lipid level of the untreated control group at the completion of testing was set to 100%, the neutral lipid level was reduced to about 80% in the group receiving a Sambucus nigra extract (300 mg/kg/day), about 87% in the group receiving a Pimpinella anisum extract (1000 mg/kg/day) and about 63% in the group receiving an Adinandra nitida extract (100 mg/kg/day).

Furthermore, the anti-obesity agents of the present invention may alter the blood levels of free fatty acids in subjects receiving the plant extracts of the present invention. Increased blood levels of free fatty acids mean that neutral lipids stored in adipocytes or neutral lipids in blood are degraded and released into blood for use as an energy source. Without being limited thereto, anti-obesity agents comprising an extract from Sambucus nigra, Pimpinella anisum or Crataegus monogyna produce an increasing effect on the blood levels of free fatty acids. Decreased blood levels of free fatty acids mean a state where the released fatty acids are taken up by the liver or other organs and their burning is further enhanced, as a result of which their blood levels are lowered. Without being limited thereto, anti-obesity agents comprising an extract from Adinandra nitida produce a decreasing effect on the blood levels of free fatty acids.

### II. Pharmaceutical Compositions and Food or Beverage Products

The present invention enables the preparation of PPAR ligand agents from extracts derived from various plants. The present invention also provides pharmaceutical compositions and food or beverage products (also collectively referred to as “the compositions of the present invention”), which comprise such a PPAR ligand agent and a pharmaceutically acceptable additive or an edible additive. The compositions of the present invention can be used for prevention and/or treatment of peroxisome proliferator-activated receptor-related diseases, depending on the in vivo distribution of PPARs and/or their physiological activity. More specifically, the compositions of the present invention are effective for prevention or amelioration of obesity, hyperlipidemia, hypertension, hyperglycemia, insulin resistance and diabetes.

In the context of the present invention, prevention of obesity is intended to mean that a subject is prevented or delayed from entering a state defined as obesity or adiposis (including visceral obesity) by the Japan Society for the Study of Obesity in the guideline manual for obesity/adiposis, second edition (published in July, 2001). Likewise, amelioration of obesity is intended to mean that a subject is shifted from a state defined as adiposis or obesity by the above society to a state defined as being within the normal range by the above society.

In the context of the present invention, prevention of hyperlipidemia is intended to mean that a subject is prevented or delayed from entering a hyperlipidemic state or a borderline state thereof, as defined by the Japan Atherosclerosis Society in the clinical guideline for arteriosclerotic disease (published in September, 2002). Likewise, amelioration of hyperlipidemia is intended to mean that a subject is shifted from a hyperlipidemic state or a borderline state thereof, as defined above, to a state defined as being within the normal range in the above guideline.

In the context of the present invention, insulin resistance refers to a state where a stimulatory effect on sugar absorption, which is the major action of insulin, is weakened in the liver, adipocytes and skeletal muscle. Prevention of insulin resistance is intended to mean that a subject is prevented or delayed from showing a worse value of insulin resistance, as assayed by the SSPG (steady-state plasma glucose) method, etc. Improvement (or amelioration) of insulin resistance is intended to mean that the above value indicative of insulin resistance is further improved. In the context of the present invention, prevention of diabetes is intended to mean that a subject is prevented or delayed from entering a diabetic state or a borderline state thereof, as defined by the Japan Diabetes Society in the diabetes treatment guideline 2002-2003 (published in May, 2002). Likewise, amelioration of diabetes is intended to mean that a subject is shifted from a diabetic state or a borderline state thereof, as defined above, to a state defined as being within the normal range in the above guideline.

The compositions of the present invention may be in any form and can be used as pharmaceutical preparations or as food or beverage products, including health foods, nutritional supplementary foods, foods with nutrient function claims, foods for specified health use, etc.

The food or beverage products of the present invention comprise one or more of the above 23 plant extracts and an additive acceptable for food or beverage purposes. Additives used for this purpose are those commonly used in food or beverage products, including vitamins (e.g., vitamin E, vitamin C), sugars, excipients, disintegrating agents, binders, lubricants, emulsifiers, isotonizing agents, buffers, solubilizers, antiseptics, stabilizers, antioxidants, coloring agents, correctives, flavorings, coagulating agents, pH adjustors, thickeners, extract powder, crude drugs, and minerals. However, additives are not limited to those listed above, as long as they do not impair the effects desired for the ligand agents of the present invention. Particularly when the food or beverage products of the present invention are supplements, it is possible to incorporate, as appropriate, vitamins (e.g., vitamin E, vitamin C) and other additives commonly blended during supplement preparation, such as emulsifiers, isotonizing agents, buffers, solubilizers, antiseptics, stabilizers, antioxidants and so on.

Food or beverage products in the context of the present invention include all food or beverage products, as exemplified by sweets (e.g., chewing gum, chocolate, candy, jelly, biscuit, cracker), frozen desserts (e.g., ice cream, glace), beverages (e.g., tea, soft drink, energy drink, beauty drink), noodles (e.g., white wheat noodle, Chinese noodle, spaghetti, instant noodle), fish cake products (e.g., fish cake (kamaboko), tube-shaped fish cake (chikuwa), puffy fish cake (hanpen)), seasonings (e.g., dressing, mayonnaise, sauce), fats and oils (e.g., margarine, butter, salad oil), bakery products, hams, soups, ready-to-eat foods and frozen foods. The intake amount of the extracts is not limited in any way. If the extracts are taken in expectation of PPAR ligand activity, more particularly of preventing and/or ameliorating obesity and obesity-associated insulin resistance, hyperlipidemia, hypertension or diabetes, their daily intake amount per adult (calculated as an extract) is 0.01 to 1000 mg/kg body weight, preferably 1 to 300 mg/kg body weight, and more preferably 2 to 20 mg/kg body weight.

The pharmaceutical compositions of the present invention comprise one or more of the above 23 plant extracts and a pharmaceutically acceptable additive. Additives used for this purpose include excipients, disintegrating agents, lubricants, binders, antioxidants, coloring agents, aggregation inhibitors, absorption promoters, solubilizers, and stabilizers. The pharmaceutical compositions may be in any form, including capsules, tablets, granules, injections, suppositories or plasters, and are particularly preferably formulated into forms intended for oral use. For use as oral compositions, the intake amount of the extracts is not limited in any way. If the extracts are taken in expectation of PPAR ligand activity, more particularly of preventing and/or ameliorating obesity and obesity-associated insulin resistance, hyperlipidemia, hypertension or diabetes, their daily dosage per adult (calculated as an extract) is 0.01 to 1000 mg/kg body weight, preferably 0.1 to 300 mg/kg body weight, and more preferably 2 to 20 mg/kg body weight, given as a single dose or in divided doses. Those skilled in the art would determine the dosage, as appropriate, by taking into account the condition of a patient (e.g., age, sex, symptom) and the intended mode of administration, etc.

“Method for Prevention and/or Treatment” and “Use for the Manufacture of a Pharmaceutical Composition”

The present invention also provides a method for prevention and/or treatment of a peroxisome proliferator-activated receptor-related disease, which comprises administering to a subject in need of such prevention and/or treatment, a pharmaceutical composition comprising the plant extract of the present invention.

The present invention further provides use of the plant extract of the present invention for the manufacture of a pharmaceutical composition used for prevention and/or treatment of a peroxisome proliferator-activated receptor-related disease.

The present invention also provides a method for prevention and/or treatment of obesity, which comprises administering to a subject in need of such prevention and/or treatment, an anti-obesity agent comprising the plant extract of the present invention.

The present invention further provides use of the plant extract of the present invention for the manufacture of an anti-obesity agent used for prevention and/or treatment of obesity.

The mode of administration, dosage and others in the “method for prevention and/or treatment” and “use for the manufacture of a pharmaceutical composition” according to the present invention are as described above for “pharmaceutical compositions.”

### BRIEF DESCRIPTION OF THE DRAWINGS

FIG. 1 shows the time course of body weight changes in animals receiving a Sambucus nigra extract. Open diamonds indicate the results of control receiving no extract, squares indicate the results of 100 mg/kg/day group, and triangles indicate the results of 300 mg/kg/day group.

FIG. 2 shows body fat levels in animals receiving Sambucus nigra. The open bar indicates the results of control receiving no extract, the horizontal hatched bar indicates the results of 100 mg/kg/day group, and the dark shaded bar indicates the results of 300 mg/kg/day group.

FIG. 3 shows blood free fatty acid levels in animals receiving Sambucus nigra. The open bar indicates the results of control receiving no extract, the horizontal hatched bar indicates the results of 100 mg/kg/day group, and the dark shaded bar indicates the results of 300 mg/kg/day group. **P<0.01 versus control

FIG. 4 shows blood neutral lipid levels in animals receiving Sambucus nigra. The open bar indicates the results of control receiving no extract, the horizontal hatched bar indicates the results of 100 mg/kg/day group, and the dark shaded bar indicates the results of 300 mg/kg/day group.

FIG. 5 shows the time course of body weight changes in animals receiving a Pimpinella anisum extract. Open diamonds indicate the results of control receiving no extract, and solid squares indicate the results of 1000 mg/kg/day group.

FIG. 6 shows body fat levels in animals receiving Pimpinella anisum. The open bar indicates the results of control receiving no extract, and the solid bar indicates the results of 1000 mg/kg/day group.

FIG. 7 shows blood free fatty acid levels in animals receiving Pimpinella anisum. The open bar indicates the results of control receiving no extract, and the solid bar indicates the results of 1000 mg/kg/day group. **P<0.01

FIG. 8 shows blood neutral lipid levels in animals receiving Pimpinella anisum. The open bar indicates the results of control receiving no extract, and the solid bar indicates the results of 1000 mg/kg/day group.

FIG. 9 shows the time course of body weight changes in animals receiving a Crataegus monogyna extract. Circles indicate the results of control receiving no extract, squares indicate the results of 100 mg/kg/day group, diamonds indicate the results of 300 mg/kg/day group, and triangles indicate the results of 1000 mg/kg/day group.

FIG. 10 shows body fat levels in animals receiving Crataegus monogyna. The open bar indicates the results of control receiving no extract, the light shaded bar indicates the results of 100 mg/kg/day group, the horizontal hatched bar indicates the results of 300 mg/kg/day group, and the solid bar indicates the results of 1000 mg/kg/day group.

FIG. 11 shows blood free fatty acid levels in animals receiving Crataegus monogyna. The open bar indicates the results of control receiving no extract, the light shaded bar indicates the results of 100 mg/kg/day group, the horizontal hatched bar indicates the results of 300 mg/kg/day group, and the solid bar indicates the results of 1000 mg/kg/day group. **P<0.01 versus control

FIG. 12 shows the time course of body weight changes in animals receiving an Adinandra nitida extract. Open diamonds indicate the results of control receiving no extract, and solid squares indicate the results of 100 mg/kg/day group.

FIG. 13 shows body fat levels in animals receiving Adinandra nitida. The open bar indicates the results of control receiving no extract, and the solid bar indicates the results of 100 mg/kg/day group.

FIG. 14 shows blood free fatty acid levels in animals receiving Adinandra nitida. The open bar indicates the results of control receiving no extract, and the solid bar indicates the results of 100 mg/kg/day group. **P<0.01

FIG. 15 shows blood neutral lipid levels in animals receiving Adinandra nitida. The open bar indicates the results of control receiving no extract, and the solid bar indicates the results of 100 mg/kg/day group.

### EXAMPLES

The present invention will now be described in more detail by way of the following examples, which are not intended to limit the scope of the invention.

Plant Materials

Table 1 shows the plants used in the examples of this specification, along with their used parts. For sample preparation, the intended part of each plant was dried and ground before use. The apparatus used for grinding was a tablet grinder, type TS-10M (TOSHO Co., Inc., Japan) or a Willey grinder, type 1029-JAS (Yoshida Seisakusho Co., Ltd., Japan).

TABLE 1

| | | Weight (g) | |
|---|---|---|---|
| | | extracted | Weight (g) extracted |
| | | in Sample | in Sample |
| | | Preparation | Preparation |
| Plant | Used part | Example 2 | Example 3 |
| Serenoa repens | Fruit | — | — |
| Acanthopanax | Root bark | — | — |
| senticosus | | | |
| Euterpe oleracea | Fruit juice | — | — |
| | powder | | |
| Elettaria | Seed | — | — |
| cardamomum | | | |
| Angelica | Root | — | — |
| archangelica | | | |
| Psidium guajava | Fruit | — | — |
| Prumus spinosa | Fruit | — | — |
| Eleagnus multiflora | Fruit | — | — |
| Ligusticum | Shoot | — | — |
| chuaxiong | | | |
| Acanthopanax | Leaf | 200 | 1.6 |
| sessiliflorus | | | |
| Cannabis sativa | Seed | — | 1.8 |
| Sambucus nigra | Flower | 250 | — |
| Adinandra nitida | Leaf | 210 | — |
| Passiflora incarnata | Shoot | 350 | — |
| Peucedanum | Shoot | 200 | — |
| japonicum | | | |
| Pimpinella anisum | Seed | 350 | — |
| Vitex agnus-castus | Fruit | 710 | — |
| Withania somnifera | Root | 350 | — |
| Calluna vulgaris | Flower | 412 | — |
| Crataegus | Fruit | 350 | — |
| monogyna | | | |
| Linum usitatissimum | Seed | 763 | 2.0 |
| Pleurotus ferulae | Fruit body | 200 | 1.7 |
| Robinia | Flower | 567 | — |
| pseudoacacia | | | |

*1 “Fruit juice powder” refers to a powder obtained by squeezing a fresh fruit and lyophilizing the resulting juice.
*2 “Terrestrial part” may optionally include shoot and flower.

### Sample Preparation Example 1

A ground product (1 g) of each plant shown in Table 1 was immersed in 10 ml (10-fold volume (W/V)) of 70 vol % ethanol and extracted at room temperature for 7 days with stirring once a day, followed by filtration to obtain an extracted solution. The extracted solution (5 ml) was concentrated and lyophilized to obtain an extract. The extract was dissolved or suspended in distilled water (1 ml). To this, water-saturated ethyl acetate (3 ml) was added and stirred, followed by centrifugation to collect the separated ethyl acetate layer. In turn, to the aqueous layer, water-saturated n-butanol (1 ml) was added and stirred, followed by centrifugation to separate into n-butanol and aqueous layers. The ethyl acetate, n-butanol and aqueous layers thus obtained were concentrated and lyophilized to give an ethyl acetate fraction, a n-butanol fraction and a water fraction.

### Sample Preparation Example 2

A ground product of Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia or Acanthopanax sessiliflorus was immersed in 70 vol % ethanol in a 10-fold volume (W/V) relative to the weight indicated in Table 1 and extracted at room temperature for 7 days with stirring once a day, followed by filtration to obtain an extracted solution. The extracted solution was concentrated and lyophilized to obtain an ethanol extract.

### Sample Preparation Example 3

A ground product of Linum usitatissimum, Pleurotus ferulae, Acanthopanax sessiliflorus or Cannabis sativa was added to distilled water in a 20-fold volume (W/V) relative to the weight indicated in Table 1 and extracted at 80° C. for 30 minutes while stirring at 10 minute intervals. Each extracted solution was cooled with water and centrifuged at 3000 rpm for 10 minutes, and the resulting supernatant was filtered. The filtrate was lyophilized to obtain a hot water extract.

### Example 1

### Measurement of PPARδ ligand activity

The ethyl acetate, n-butanol and water fractions of each plant obtained in Sample Preparation Example 1 were measured for their PPARδ ligand activity in the following manner.

HepG2 cells (human liver cancer-derived cultured cells) were seeded in 96-well culture plates at 1×10⁴ cells/well and cultured at 37° C. under 5% CO₂ for 24 hours. The medium used was RPMI1640 (Nissui Pharmaceutical Co., Ltd., Japan) containing 10% FBS (fetal bovine serum; Equitech-Bio) and 0.3 g/L L-Glutamine (Nissui Pharmaceutical Co., Ltd., Japan). These cells were washed with OPTI-MEM (Gibco) and then transfected with pBIND/GAL4::mPPARδ and pG51uc (Promega). It should be noted that pBIND/GAL4::mPPARδ is a plasmid obtained by inserting the mouse PPARδ gene into a yeast transcription factor GAL4 fusion protein expression plasmid, pBIND (Promega), while pG51uc is a reporter plasmid designed to have 5 copies of a GAL4-responsive sequence (UAS) upstream of the luciferase gene (which can be prepared as described in, e.g., Cell, 1995, vol. 83, pp. 803-812).

At about 24 hours after transfection, the medium was replaced with another medium containing a plant extract, followed by culturing for 24 hours. As a vehicle control, DMSO was used and added in 1/100 volume to the medium. After washing with phosphate-buffered saline (PBS—), the cells were lysed with a cytolysis solution (Cell Culture Lysis Reagent: Promega) and measured for the luminescence intensity of luciferase with a multilabel counter (Wallac) in the presence of a Luciferase Assay Reagent (Promega). On the other hand, a synthetic PPARδ agonist, L-165041 (Sigma, 25 μl), was used as a positive control. For assessment of ligand activity, the ratio of luminescence intensity in a sample to that in the control (vehicle control) was defined as the luminescence ratio of PPARδ ligand activity in the sample, and a sample showing a value of 1.3 or greater was determined as having PPARδ ligand activity. Table 2 shows the sample concentration (sample concentration in the medium) used for activity measurement, along with the ligand activity (luminescence ratio) obtained for each sample.

TABLE 2
PPARδ ligand activity

| | | PPARδ | | |
|---|---|---|---|---|
| | | Sample | Ligand activity | Luminescence |
| | | concentration | (luminescence | ratio in |
| Plant name | Extract, fraction | (μg/mL) | ratio) | L-165041 |
| Serenoa repens | n-Butanol fraction | 100/3 | 2.0 | 2.8 |
| Serenoa repens | Water fraction | 100/3 | 1.4 | 2.8 |
| Acanthopanax | n-Butanol fraction | 100/27 | 1.3 | 3.1 |
| senticosus | | | | |
| Acanthopanax | Water fraction | 100/3 | 1.4 | 2.8 |
| senticosus | | | | |
| Euterpe oleracea | n-Butanol fraction | 100/3 | 1.5 | 2.7 |
| Euterpe oleracea | Water fraction | 100/9 | 1.5 | 3.3 |
| Elettaria | Ethyl acetate | 100/3 | 1.5 | 3.3 |
| cardamomum | fraction | | | |
| Elettaria | n-Butanol fraction | 100/27 | 1.9 | 3.2 |
| cardamomum | | | | |
| Angelica | n-Butanol fraction | 100/81 | 1.9 | 2.8 |
| archangelica | | | | |
| Psidium guajava | n-Butanol fraction | 100/27 | 1.8 | 2.8 |
| Prumus spinosa | n-Butanol fraction | 100/3 | 1.9 | 2.8 |
| Eleagnus multiflora | n-Butanol fraction | 100/81 | 1.9 | 2.8 |
| Ligusticum | n-Butanol fraction | 100/27 | 1.4 | 3.7 |
| chuaxiong | | | | |
| Acanthopanax | Water fraction | 100/27 | 1.7 | 4.3 |
| sessiliflorus | | | | |
| Cannabis sativa | n-Butanol fraction | 100/27 | 2.2 | 3.2 |
| Cannabis sativa | Water fraction | 100/27 | 2.2 | 2.8 |
| Sambucus nigra | Ethyl acetate | 100/3 | 1.7 | 3.9 |
| | fraction | | | |
| Sambucus nigra | n-Butanol fraction | 100/3 | 1.8 | 3.2 |
| Adinandra nitida | Ethyl acetate | 100/81 | 2.4 | 2.6 |
| | fraction | | | |
| Adinandra nitida | n-Butanol fraction | 100/81 | 3.2 | 2.8 |
| Passiflora incarnata | n-Butanol fraction | 100/81 | 2.0 | 2.8 |
| Peucedanum | n-Butanol fraction | 100/81 | 1.9 | 2.8 |
| japonicum | | | | |
| Pimpinella anisum | n-Butanol fraction | 100/81 | 1.8 | 2.8 |
| Vitex agnus-castus | n-Butanol fraction | 100/9 | 1.7 | 2.8 |
| Withania somnifera | Water fraction | 100/81 | 1.6 | 3.8 |
| Calluna vulgaris | Ethyl acetate | 100/3 | 1.7 | 5.8 |
| | fraction | | | |
| Calluna vulgaris | Water fraction | 100/27 | 1.5 | 3.7 |
| Crataegus | Water fraction | 100/81 | 1.8 | 4.4 |
| monogyna | | | | |
| Linum usitatissimum | Water fraction | 100/81 | 1.9 | 4.3 |
| Pleurotus ferulae | Water fraction | 100/3 | 2.3 | 3.5 |
| Robinia | Water fraction | 100/81 | 1.5 | 3.3 |
| pseudoacacia | | | | |

### Example 2

### Measurement of PPARα Ligand Activity

The same procedure as shown in Example 1 was repeated to measure PPARα ligand activity, except that pBIND/GAL4::mPPARα was used as a fusion protein expression plasmid instead of pBIND/GAL4::mPPARδ. It should be noted that pBIND/GAL4::mPPARα is a plasmid obtained by inserting the mouse PPARα gene into pBIND (Promega). On the other hand, a synthetic PPARα agonist, WY-14643 (Sigma, 25 μM), was used as a positive control. Table 3 shows the sample concentration (sample concentration in the medium) used for activity measurement, along with the ligand activity (luminescence ratio) obtained for each sample.

TABLE 3
PPARα ligand activity

| | | PPARα | | |
|---|---|---|---|---|
| | Solvent used | Sample | | Luminescence |
| | for | concentration | Ligand activity | ratio in |
| Plant name | fractionation | (μg/mL) | (luminescence ratio) | WY-14643 |
| Elettaria | Water fraction | 100/3 | 1.5 | 7.6 |
| cardamomum | | | | |
| Linum | Ethyl acetate | 100/1 | 2.8 | 4.2 |
| usitatissimum | fraction | | | |
| Linum | n-Butanol | 100/3 | 1.5 | 4.4 |
| usitatissimum | fraction | | | |

### Example 3

### Measurement of PPARγ Ligand Activity

CHO cells used for measurement of PPARγ ligand activity were obtained in the following manner.

A PPAR reporter plasmid (pPPRE-Luc) was derived from a reporter plasmid, pGL3 (Promega), which contains the SV40 promoter gene and the firefly luciferase gene by being modified to have 3 copies of a PPAR-responsive sequence (PPRE) upstream of the SV40 promoter gene. A PPARγ expression plasmid (pKD-rPPARγ) was obtained by inserting the rat PPARγ gene into an SV40 promoter-driven expression plasmid for mammalian cells. pPPRE-Luc and pKD-rPPARγ were co-transfected into CHO(dhfr-) cells (dihydrofolate reductase-deficient cell line of Chinese hamster ovary cells) using Lipofectamine (Invitorgen). The cells were cultured in D-MEM medium (GIBCO) almost free from thymidine and containing dialyzed fetal bovine serum (GIBCO) to thereby obtain a stable transformant (CHO/PPARγ/PPRE) retaining pPPRE-Luc and pKD-rPPARγ.

The above cells were seeded in 96-well culture plates at 1×10⁴ cells/well and cultured at 37° C. under 5% CO₂ for 24 hours. The medium used was DMEM (Nissui Pharmaceutical Co., Ltd., Japan) containing 10% FBS (fetal bovine serum; Equitech-Bio), 0.3 g/L L-Glutamine (Nissui Pharmaceutical Co., Ltd., Japan) and 10 ml/L nonessential amino acid solution for MEM (Dainippon Sumitomo Pharma Co., Ltd., Japan). After culturing for 24 hours, the medium was replaced with another medium containing a plant extract, and culture was continued for an additional 24 hours. As a vehicle control, DMSO was used and added in 1/100 volume to the medium. After washing with phosphate-buffered saline (PBS-), the cells were lysed with a cytolysis solution (Cell Culture Lysis Reagent: Promega) and measured for the luminescence intensity of luciferase with a multilabel counter (Wallac) in the presence of a Luciferase Assay Reagent (Promega). On the other hand, a synthetic PPARγ agonist, Ciglitizone (Sigma, 25 μM), was used as a positive control. Table 4 shows the sample concentration (sample concentration in the medium) used for activity measurement, along with the ligand activity (luminescence ratio) obtained for each sample.

TABLE 4
PPARγ ligand activity

| | | PPARγ | | |
|---|---|---|---|---|
| | | Sample | Ligand activity | Luminescence |
| | | concentration | (luminescence | ratio in |
| Plant name | Extract, fraction | (μg/mL) | ratio) | Ciglitizone |
| Acanthopanax | Ethyl acetate fraction | 100/1 | 1.3 | 2.6 |
| senticosus | | | | |
| Eleagnus multiflora | Ethyl acetate fraction | 100/3 | 1.9 | 2.2 |
| Cannabis sativa | Ethyl acetate fraction | 100/3 | 1.4 | 2.5 |
| Sambucus nigra | Ethyl acetate fraction | 100/3 | 1.8 | 2.6 |
| Adinandra nitida | Ethyl acetate fraction | 100/3 | 2.0 | 2.7 |
| Adinandra nitida | n-Butanol fraction | 100/1 | 1.4 | 3.0 |
| Pimpinella anisum | Ethyl acetate fraction | 100/1 | 2.2 | 2.4 |
| Calluna vulgaris | Ethyl acetate fraction | 100/3 | 2.1 | 2.6 |

### Example 4

### Measurement of PPARγ Ligand Activity in Ethanol Extracts

The ethanol extract of each plant obtained in Sample Preparation Example 2 was measured for its PPARγ ligand activity in the same manner as shown in Example 1. Table 5 shows the sample concentration (sample concentration in the medium) used for activity measurement, along with the ligand activity (luminescence ratio) obtained for each sample.

TABLE 5
PPARδ ligand activity in ethanol extracts

| | PPARδ | | |
|---|---|---|---|
| | Sample | Ligand activity | |
| | concentration | (luminescence | Luminescence |
| Plant | (μg/mL) | ratio) | ratio in L-165041 |
| Sambucus nigra | 100/9 | 1.4 | 4.5 |
| Adinandra nitida | 100/9 | 1.7 | 4.7 |
| Passiflora incarnata | 100/9 | 1.4 | 4.5 |
| Peucedanum | 100/27 | 1.5 | 4.9 |
| japonicum | | | |
| Pimpinella anisum | 100/81 | 2.1 | 4.9 |
| Vitex agnus-castus | 100/3 | 1.8 | 6.1 |
| Withania somnifera | 100/81 | 1.9 | 6.1 |
| Calluna vulgaris | 100/27 | 1.9 | 6.2 |
| Crataegus | 100/9 | 1.8 | 6.2 |
| monogyna | | | |
| Linum usitatissimum | 100/81 | 1.5 | 5.2 |
| Pleurotus ferulae | 100/9 | 1.7 | 5.2 |
| Robinia | 100/81 | 1.5 | 4.8 |
| pseudoacacia | | | |
| Acanthopanax | 100/9 | 1.7 | 4.7 |
| sessiliflorus | | | |

### Example 5

### Measurement of PPARδ Ligand Activity in Hot Water Extracts

The hot water extract of each plant obtained in Sample Preparation Example 3 was measured for its PPARδ ligand activity in the same manner as shown in Example 1. Table 6 shows the sample concentration (sample concentration in the medium) used for activity measurement, along with the ligand activity (luminescence ratio) obtained for each sample.

TABLE 6
PPARδ ligand activity in hot water extracts

| | PPARδ | | |
|---|---|---|---|
| | Sample | Ligand activity | |
| | concentration | (luminescence | Luminescence ratio |
| Plant | (μg/mL) | ratio) | in L-165041 |
| Linum | 100/9 | 1.9 | 5.8 |
| usitatissimum | | | |
| Pleurotus ferulae | 100/9 | 1.5 | 4.5 |
| Acanthopanax | 100/9 | 1.5 | 3.8 |
| sessiliflorus | | | |
| Cannabis sativa | 100/3 | 2.0 | 6.2 |

As shown in Examples 1 to 5, each plant extract was found to have PPAR ligand activity. The detailed results are as follows.

(1) In Sambucus nigra, its ethyl acetate fraction was found to have both PPARδ and PPARγ ligand activities, and its n-butanol fraction was found to have PPARδ ligand activity. Moreover, its ethanol extract was found to have PPARδ activity.

(2) In Adinandra nitida, its ethyl acetate fraction was found to have both PPARδ and PPARγ ligand activities, and its n-butanol fraction was also found to have both PPARδ and PPARγ ligand activities. Moreover, its ethanol extract was found to have PPARδ activity.

(3) In Passiflora incarnata, its n-butanol fraction was found to have PPARδ ligand activity, and its ethanol extract was found to have PPARδ activity.

(4) In Peucedanum japonicum, its n-butanol fraction was found to have PPARδ ligand activity, and its ethanol extract was found to have PPARδ activity.

(5) In Pimpinella anisum, its ethyl acetate fraction was found to have PPARγ ligand activity, and its n-butanol fraction was found to have PPARδ ligand activity. Moreover, its ethanol extract was found to have PPARδ activity.

(6) In Vitex agnus-castus, its n-butanol fraction was found to have PPARδ ligand activity, and its ethanol extract was found to have PPARδ activity.

(7) In Withania somnifera, its water fraction was found to have PPARδ ligand activity, and its ethanol extract was found to have PPARδ activity.

(8) In Calluna vulgaris, its ethyl acetate fraction was found to have both PPARδ and PPARγ ligand activities, and its water fraction was found to have PPARδ ligand activity. Moreover, its ethanol extract was found to have PPARδ activity.

(9) In Crataegus monogyna, its water fraction was found to have PPARδ ligand activity, and its ethanol extract was found to have PPARδ activity.

(10) In Linum usitatissimum, its ethyl acetate fraction was found to have PPARα ligand activity, its n-butanol fraction was also found to have PPARα ligand activity, and its water fraction was found to have PPARδ ligand activity. Moreover, its ethanol extract was found to have PPARδ activity, and its hot water extract was also found to have PPARδ activity.

(11) In Pleurotus ferulae, its water fraction was found to have PPARδ ligand activity. Moreover, its ethanol extract was found to have PPARδ activity, and its hot water extract was also found to have PPARδ activity.

(12) In Robinia pseudoacacia, its water fraction was found to have PPARδ ligand activity, and its ethanol extract was found to have PPARδ activity.

(13) In Acanthopanax sessiliflorus, its water fraction was found to have PPARδ ligand activity. Moreover, its ethanol extract was found to have PPARδ activity, and its hot water extract was also found to have PPARδ activity.

(14) In Cannabis sativa, its ethyl acetate fraction was found to have PPARγ ligand activity, its n-butanol fraction was found to have PPARδ ligand activity, and its water fraction was also found to have PPARδ ligand activity. Moreover, its hot water extract was found to have PPARδ activity.

(15) In Elettaria cardamomum, its ethyl acetate fraction was found to have PPARδ ligand activity, and its n-butanol fraction was also found to have PPARδ ligand activity. Moreover, its water fraction was found to have PPARα ligand activity.

(16) In Angelica archangelica, its n-butanol fraction was found to have PPARδ ligand activity.

(17) In Psidium guajava, its n-butanol fraction was found to have PPARδ ligand activity.

(18) In Prumus spinosa, its n-butanol fraction was found to have PPARδ ligand activity.

(19) In Serenoa repens, its n-butanol fraction was found to have PPARδ ligand activity, and its water fraction was also found to have PPARδ ligand activity.

(20) In Eleagnus multiflora, its ethyl acetate fraction was found to have PPARγ ligand activity, and its n-butanol fraction was found to have PPARδ ligand activity.

(21) In Ligusticum chuaxiong, its n-butanol fraction was found to have PPARδ ligand activity.

(22) In Acanthopanax senticosus, its ethyl acetate fraction was found to have PPARγ ligand activity, and its n-butanol fraction was found to have PPARδ ligand activity. Moreover, its water fraction was also found to have PPARδ ligand activity.

(23) In Euterpe oleracea, its n-butanol fraction was found to have PPARδ ligand activity, and its water fraction was also found to have PPARδ ligand activity.

### Example 6

### Test for Anti-Obesity Effect

### (Test Procedures)

Plant extracts of Sambucus nigra, Adinandra nitida, Pimpinella anisum and Crataegus monogyna were tested for their anti-obesity effect in the following manner. The animals used were male ddY mice at 6 weeks of age (Kiwa Laboratory Animals Co., Ltd., Japan), which were acclimated and fed with a standard feed (CE-2; CLEA Japan, Inc.) for one week before being administered with a sample. For sample administration, a plant extract of Sambucus nigra, Adinandra nitida, Pimpinella anisum or Crataegus monogyna prepared in the same manner as shown in Sample Preparation Example 2 was suspended in purified water containing 3% gum arabic, and given to the mice by forced oral administration at the dosage described later, once a day in the morning, 12 times in total over 17 days. On the other hand, the control group was administered with purified water containing 3% gum arabic alone. The feed used during sample administration was a high-fat diet containing 45 kcal % fat (D12415, Research Diet, Inc.), and the mice were allowed to take water ad libitum during the test period.

At 16 days after initiation of administration, which was the day before dissection, the mice were measured for their body fat levels using an X-ray CT system for laboratory animals (Latheta LCT-100, Aloka Co., Ltd., Japan). On the day of dissection, the mice received no administration and were fasted for 4 hours, followed by collecting their entire blood under ether anesthesia from the abdominal vena cava. After centrifugation, plasma was obtained and stored at −80° C. At a later date, neutral lipid and free fatty acid levels in blood were measured using an automatic analyzer (Hitachi 7070, Hitachi, Ltd., Japan).

6-1 Sambucus nigra

### (Results)

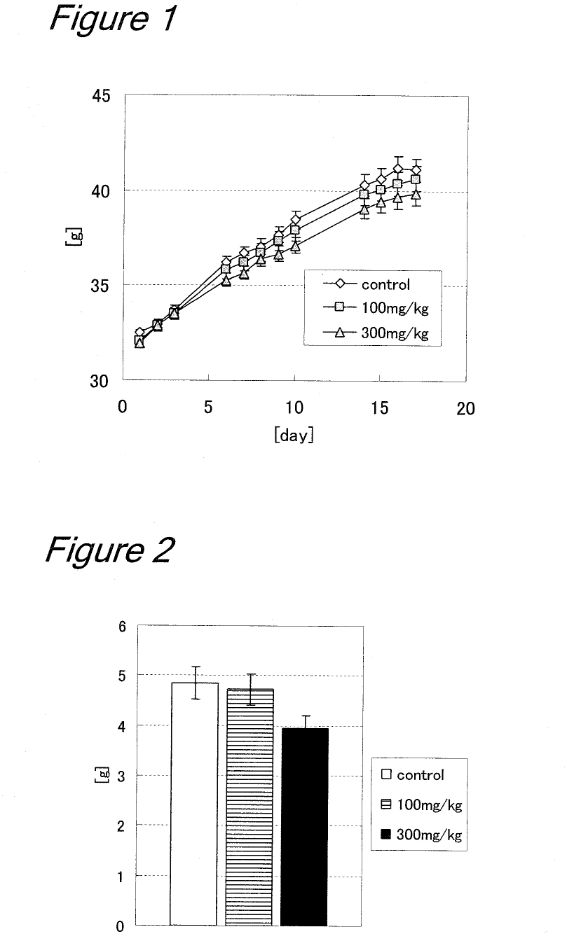

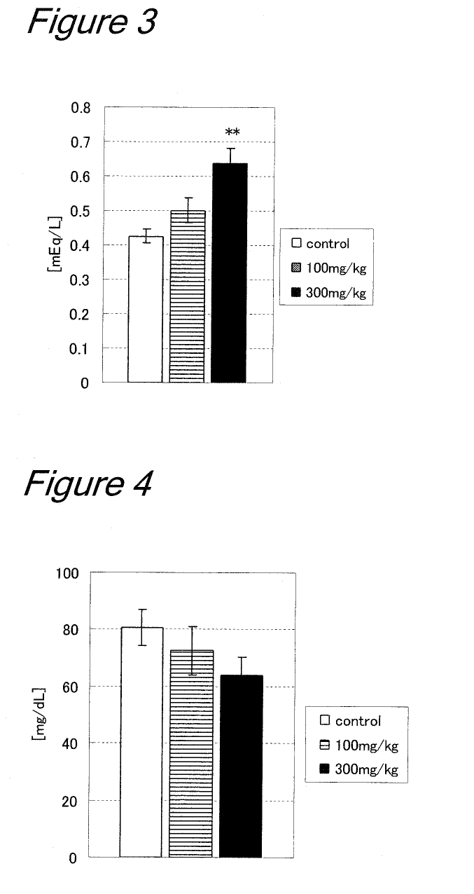

FIG. 1 shows the time course of body weight changes during the test period in the mice receiving a Sambucus nigra extract at a dose of 100 or 300 mg/kg/day (in a volume of 10 mL/kg), while FIG. 2 shows body fat levels in these mice at day 16 of administration. Likewise, FIGS. 3 and 4 show blood free fatty acid levels and blood neutral lipid levels in the mice at the completion of the test, respectively.

As shown in FIGS. 1 to 4, the Sambucus nigra extract suppressed both body weight gain and body fat accumulation in a dose-dependent manner. Moreover, this extract caused a significant increase in blood free fatty acid levels, whereas it caused a decrease in neutral lipid levels.

6-2 Pimpinella anisum

### (Results)

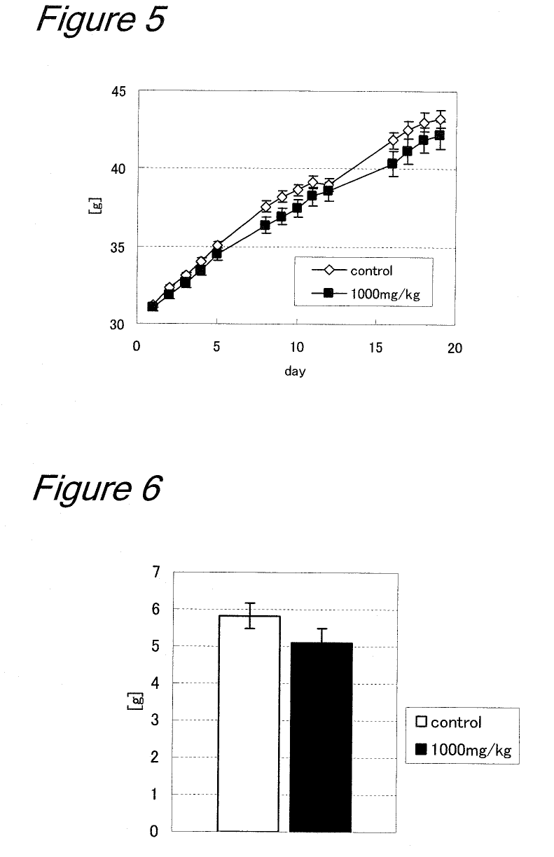

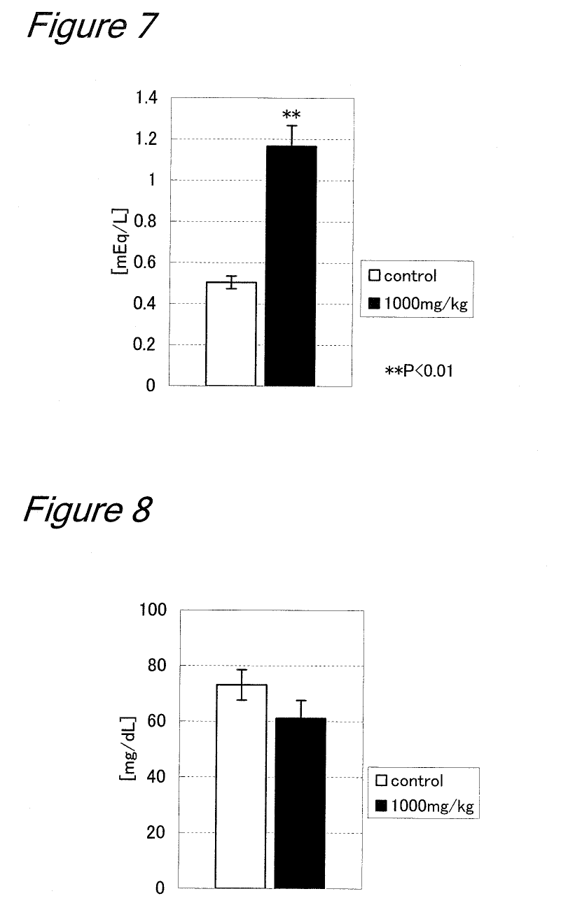

FIG. 5 shows the time course of body weight changes during the test period in the mice receiving a Pimpinella anisum extract at a dose of 1000 mg/kg/day (in a volume of 10 mL/kg), while FIG. 6 shows body fat levels in these mice at day 16 of administration. Likewise, FIGS. 7 and 8 show blood free fatty acid levels and blood neutral lipid levels in the mice at the completion of the test, respectively.

As shown in FIGS. 5 to 8, administration of the Pimpinella anisum extract suppressed both body weight gain and body fat accumulation. Moreover, this extract caused a significant increase in blood free fatty acid levels, whereas it caused a decrease in neutral lipid levels.

6-3 Crataegus monogyna

### (Results)

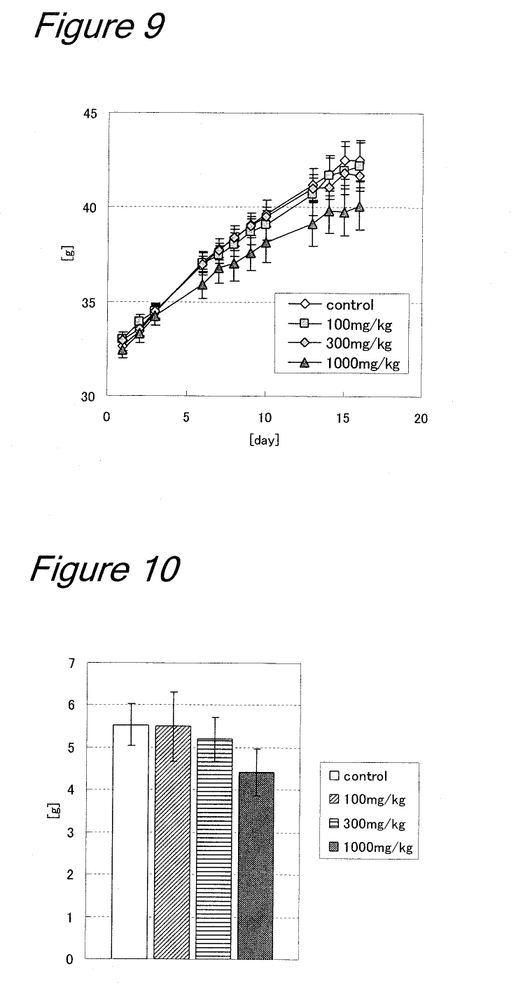

FIG. 9 shows the time course of body weight changes during the test period in the mice receiving a Crataegus monogyna extract at a dose of 100, 300 or 1000 mg/kg/day (in a volume of 10 mL/kg), while FIG. 10 shows body fat levels in these mice at day 16 of administration. Likewise, FIG. 11 shows the results measured for blood free fatty acid levels at the completion of the test.

As shown in FIGS. 9 to 11, the Crataegus monogyna extract suppressed both body weight gain and body fat accumulation in a dose-dependent manner. Moreover, this extract caused a significant dose-dependent increase in blood free fatty acid levels.

6-4 Adinandra nitida

### (Results)

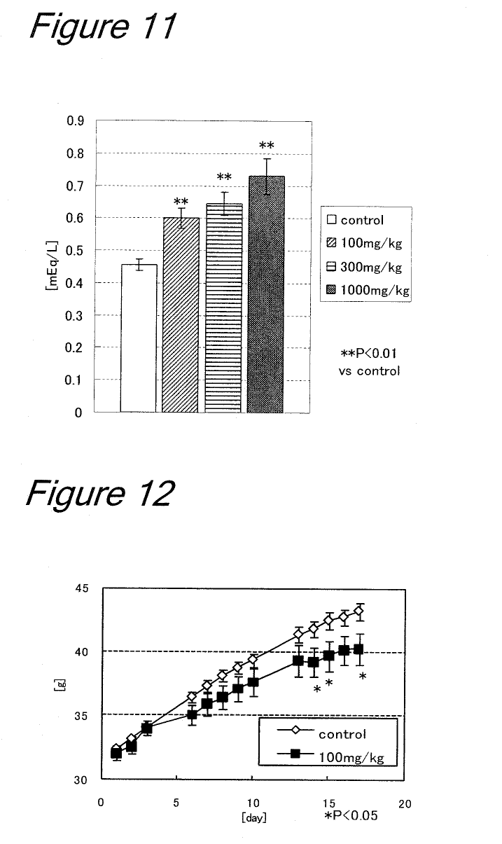

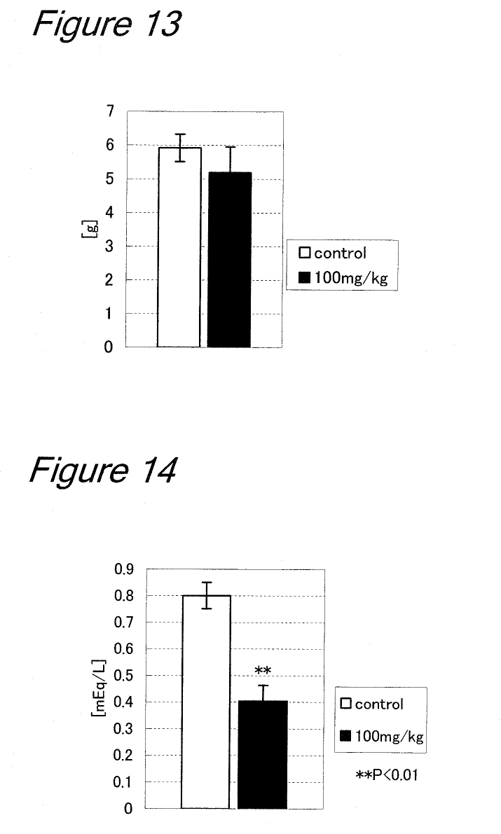

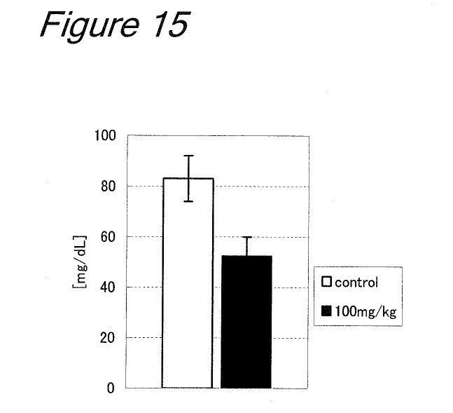

FIG. 12 shows the time course of body weight changes during the test period in the mice receiving an Adinandra nitida extract at a dose of 100 mg/kg/day (in a volume of 10 mL/kg), while FIG. 13 shows body fat levels in these mice at day 16 of administration. Likewise, FIGS. 14 and 15 show blood free fatty acid levels and blood neutral lipid levels in the mice at the completion of the test, respectively.

Administration of the Adinandra nitida extract resulted in a significant suppression of body weight gain and also suppressed body fat accumulation. Moreover, this extract caused a significant decrease in both free fatty acid levels and neutral lipid levels.

1. A ligand agent for a peroxisome proliferator-activated receptor (PPAR), which comprises, as an active ingredient, an extract of one or more plants selected from the following:
Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

2. The ligand agent according to claim 1, wherein the PPAR is PPARδ.

3. The ligand agent according to claim 1, wherein the plant is one or more selected from Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

4. The ligand agent according claim 1, wherein the extract is an alcohol extract or an aqueous alcohol extract.

5. The ligand agent according to claim 4, wherein the alcohol is ethanol.

6. The ligand agent according to claim 1, wherein the plant is one or more selected from Linum usitatissimum, Pleurotus ferulae, Acanthopanax sessiliflorus, and Cannabis sativa.

7. The ligand agent according to claim 1, wherein the extract is a hot water extract.

8. A pharmaceutical composition used for prevention and/or treatment of a peroxisome proliferator-activated receptor-related disease, which comprises an extract of one or more plants selected from the following:
Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus
and a pharmaceutically acceptable additive.

9. The pharmaceutical composition according to claim 8, wherein the peroxisome proliferator-activated receptor-related disease is one or more diseases selected from obesity, hyperlipidemia, hypertension and diabetes.

10. A food or beverage product, which comprises an extract of one or more plants selected from the following:
Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus
and an additive acceptable for food or beverage purposes.

11. The food or beverage product according to claim 10, which is used for prevention and/or amelioration of a peroxisome proliferator-activated receptor-related disease.

12. An anti-obesity agent, which comprises an extract of one or more plants selected from the group consisting of Sambucus nigra, Pimpinella anisum, Crataegus monogyna and Adinandra nitida as an active ingredient.

13. A method for prevention and/or treatment of a peroxisome proliferator-activated receptor-related disease, which comprises administering to a subject in need of such prevention and/or treatment, a pharmaceutical composition comprising an extract of one or more plants selected from the following:
Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

14. Use for the manufacture of a pharmaceutical composition used for prevention and/or treatment of a peroxisome proliferator-activated receptor-related disease, wherein the pharmaceutical composition comprises an extract of one or more plants selected from the following:
Serenoa repens, Acanthopanax senticosus, Euterpe oleracea, Elettaria cardamomum, Angelica archangelica, Psidium guajava, Prumus spinosa, Eleagnus multiflora, Ligusticum chuaxiong, Cannabis sativa, Sambucus nigra, Adinandra nitida, Passiflora incarnata, Peucedanum japonicum, Pimpinella anisum, Vitex agnus-castus, Withania somnifera, Calluna vulgaris, Crataegus monogyna, Linum usitatissimum, Pleurotus ferulae, Robinia pseudoacacia, and Acanthopanax sessiliflorus.

15. A method for prevention and/or treatment of obesity, which comprises administering to a subject in need of such prevention and/or treatment, an anti-obesity agent comprising an extract of one or more plants selected from the group consisting of Sambucus nigra, Pimpinella anisum, Crataegus monogyna and Adinandra nitida.

16. Use for the manufacture of an anti-obesity agent used for prevention and/or treatment of obesity, wherein the anti-obesity agent comprises an extract of one or more plants selected from the group consisting of Sambucus nigra, Pimpinella anisum, Crataegus monogyna and Adinandra nitida.
